(19) United States
(12) Patent Application Publication
(10) Pub. No.: US 2026/0294433 A1
(43) Pub. Date: Oct. 1, 2026

(54) SURGICAL STAPLING DEVICE WITH ARTICULATING TOOL ASSEMBLY

(71) Applicant: Covidien LP, Mansfield, MA (US)

(72) Inventor: David M. FARASCIONI, Ridgefield, CT (US)

(21) Appl. No.: 19/478,944

(22) PCT Filed: Apr. 25, 2024
(86) PCT No.: PCT/IB2024/054043

§ 371 Date: Oct. 27, 2025

Related U.S. Application Data
(60) Provisional application No. 63/462,583, filed on Apr. 28, 2023.

(51) Int. Cl. A61B 17/072; A61B 17/00

(52) U.S. Cl. CPC A61B 17/07207; A61B 2017/00477

(57) ABSTRACT

A surgical stapling device includes an adapter assembly defining a longitudinal axis, a tool assembly that is pivotably coupled to the adapter assembly about an articulation axis that is transverse to the longitudinal axis, and a drive mechanism that includes a drive beam that is positioned distally of the articulation axis.

This Application claims priority from U.S. Provisional Patent Application 63/462,583, filed 28 Apr. 2023, the entire content of which is incorporated herein by reference.

### FIELD

This disclosure is generally related to surgical stapling devices and, more particularly, to surgical stapling devices having articulating tool assemblies.

### BACKGROUND

Endoscopic stapling devices that include an elongated body and a tool assembly supported on the elongated body are well known. Typically, the tool assembly is coupled to a distal portion of the elongate body by a pivot member that defines an articulation axis that is transverse to a longitudinal axis of the elongate body to facilitate pivotable movement of the tool assembly between articulated and non-articulated positions. Such stapling devices include a drive assembly having a flexible drive beam that can bend about the articulation axis when the tool assembly is fired to eject staples from the tool assembly and cut tissue.

To gain better access to tissue within a body cavity during an endoscopic surgical procedure, articulation of the tool assembly over a range of zero to ninety-degrees is desirable. However, advancing the drive assembly about the articulation axis to fire the stapling device with the tool assembly articulated to such high degrees greatly increases the forces required to fire the stapling device.

A continuing need exists in the art for a stapling device that can minimize the firing forces required to fire the stapling device when the tool assembly of the stapling device is in an articulated position.

### SUMMARY

This disclosure generally relates to a surgical stapling device that includes an adapter assembly, a drive mechanism, and a tool assembly that is pivotably supported on a distal portion of the adapter assembly and pivotable about an articulation axis between a non-articulated position and articulated positions. The drive mechanism includes a distal drive assembly that is supported on the tool assembly to articulate with the tool assembly. The distal drive assembly includes a clamp member and one or more coiled bands that are connected to the clamp member and movable from a coiled configuration to an extended configuration to advance the clamp member in relation to the tool assembly.

Aspects of the disclosure are directed to a surgical stapling device that includes an adapter assembly, a tool assembly, and a distal drive assembly. The adapter assembly has a distal portion that supports a proximal drive assembly that has a rotatable drive gear and a proximal gear that is engaged with the drive gear. The tool assembly includes an anvil and a cartridge assembly that are coupled to each other to facilitate movement of the tool assembly between an unclamped position and a clamped position. The tool assembly includes a proximal portion and a distal portion. The distal drive assembly is secured to the proximal portion of the tool assembly and includes a pivot member, a sprocket, a sprocket gear, a coiled band having a distal portion, and a clamp member secured to the distal portion of the coiled band. The coiled band is movable, in response to rotation of the sprocket, between a coiled configuration and an extended configuration to move the clamp member in relation to the tool assembly between retracted and advanced positions.

Other aspects of the disclosure are directed to a surgical stapling device including a handle assembly, an adapter assembly, a tool assembly, and a distal drive assembly. The adapter assembly has a proximal portion and a distal portion, and the proximal portion of the adapter assembly is coupled to the handle assembly. The tool assembly includes an anvil and a cartridge assembly that are coupled to each other to facilitate movement of the tool assembly between an unclamped position and a clamped position. The distal drive assembly is secured to a proximal portion of the tool assembly and includes a clamp member, a coiled band, and a pivot member that is pivotably coupled to the distal portion of the adapter assembly about an articulation axis. The pivot member defines a longitudinal slot, and the coiled band is supported on the pivot member and includes a distal portion that extends through the longitudinal slot. The coiled band is movable between a coiled configuration and an extended configuration to move the clamp member in relation to the tool assembly between retracted and advanced positions.

Other aspects of the disclosure are directed to a surgical stapling device including an adapter assembly, a tool assembly, a distal drive assembly, and first and second articulation cables. The adapter assembly has a proximal portion and a distal portion that supports a proximal drive assembly that has a rotatable drive gear and a proximal gear. The tool assembly includes an anvil and a cartridge assembly that are coupled to each other to facilitate movement of the tool assembly between an unclamped position and a clamped position. The distal drive assembly includes a pivot member that is secured to a proximal portion of the tool assembly and includes a sprocket having fingers, first and second coiled bands, and a clamp member that is secured to the first and second coiled bands. The sprocket is operatively coupled to the proximal gear of the proximal drive assembly. The first and second coiled bands define longitudinally aligned openings that sequentially receive the fingers such that rotation of the sprocket moves the first and second coiled bands between a coiled configuration and an extended configuration to move the clamp member in relation to the tool assembly between retracted and advanced positions. The proximal drive assembly is supported on the distal portion of the adapter assembly and includes a housing portion that defines circular cutouts. The pivot member defines circular pivots that are received in the circular cutouts to pivotably couple the pivot member to the proximal drive assembly. The first and second articulation cables extend through channels in the proximal drive assembly and the pivot member such that tension in the first and second articulation cables retains the circular pivots within the circular cutouts.

In aspects of the disclosure, the sprocket includes fingers, and the coiled band defines openings that receive the fingers of the sprocket such that rotation of the sprocket in a first direction causes advancement of the distal portion of the coiled band to move the clamp member in relation to the tool assembly.

In some aspects of the disclosure, the coiled band includes first and second coiled bands.

In certain aspects of the disclosure, the pivot member defines a longitudinal slot that is longitudinally aligned with the distal portion of the first and second coiled bands.

In aspects of the disclosure, the pivot member is pivotably coupled to the proximal drive assembly about an articulation axis such that the coiled band pivots about the articulation axis with the pivot member.

In some aspects of the disclosure, the stapling device includes first and second articulation cables that extend through the adapter assembly and are coupled to the pivot member to pivot the tool assembly about the articulation axis.

In certain aspects of the disclosure, the proximal drive assembly includes a distal housing portion that defines circular cutouts, and the pivot member includes circular pivots that are received within the circular cutouts to pivotably couple the pivot member to the proximal drive assembly.

In aspects of the disclosure, the first and second articulation cables extend through channels in the proximal drive assembly and the pivot member, and tension in the first and second articulation cables retains the circular pivots within the circular cutouts.

In some aspects of the disclosure, the proximal drive assembly includes a proximal gear that is engaged with the drive gear and the sprocket gear such that rotation of the proximal gear causes rotation of the sprocket gear.

In certain aspects of the disclosure, the first and second bands are positioned side-by-side as the distal portions of the first and second bands pass through the longitudinal slot of the pivot member to form a substantially rigid drive beam.

In aspects of the disclosure, the stapling device includes a handle assembly, and the proximal portion of the adapter assembly is coupled to the handle assembly.

In some aspects of the disclosure, the distal drive assembly pivots with the tool assembly between the non-articulated position and the articulated positions.

Other aspects, features, and advantages will be apparent from the description, the drawings, and the claims that follow.

### BRIEF DESCRIPTION OF DRAWINGS

Various aspects and features of the disclosure are described with reference to the drawings wherein like numerals designate identical or corresponding elements in each of the several views wherein:

FIG. 1 is a side perspective view of a stapling device according to aspects of the disclosure with a tool assembly of the stapling device in an unclamped, non-articulated position;

FIG. 2 is an exploded view of a distal portion of the stapling device shown in FIG. 1 including the tool assembly and a drive mechanism;

FIG. 3 is an enlarged view of the indicated area of detail shown in FIG. 2;

FIG. 3A is a perspective view from the distal end of a pivot member of the stapling device shown in FIG. 2;

FIG. 3B is a cross-sectional view taken along section line 3B-3B of FIG. 3A;

FIG. 4 is an enlarged perspective view from one side of portions of a distal drive assembly of the drive mechanism of the stapling device shown in FIG. 1 including a clamp member, coiled bands, and a sprocket;

FIG. 5 is an enlarged perspective view from an opposite side of the portions of a distal drive assembly of the drive mechanism shown in FIG. 4;

FIG. 6 is a side perspective view of the distal portion of an adapter assembly and a proximal portion of the tool assembly of the stapling device shown in FIG. 1 with an outer tube of the adapter assembly and the tool assembly shown in phantom;

FIG. 7 is a cross-sectional view taken along section line 7-7 of FIG. 6;

FIG. 8 is a side perspective view of the distal portion of the adapter assembly and the drive mechanism of the stapling device shown in FIG. 1 with the distal drive assembly of the drive mechanism in a non-articulated position;

FIG. 9 is an enlarged view of the indicated area of detail shown in FIG. 1;

FIG. 10 is a cross-sectional view taken along section line 10-10 of FIG. 7;

FIG. 11 is a side perspective view of the distal portion of the stapling device shown in FIG. 1 with the outer tube of the adapter assembly removed and the tool assembly in an articulated, unclamped position;

FIG. 12 is a cross-sectional view taken along section line 12-12 of FIG. 11;

FIG. 13 is a cross-sectional view taken through the distal portion of the adapter assembly shown in FIG. 11 with the tool assembly in the articulated position and fired position and the drive mechanism in a drive advanced position; and

FIG. 14 is an enlarged view of the indicated area of detail shown in FIG. 13.

### DETAILED DESCRIPTION

The disclosed surgical stapling device will now be described in detail with reference to the drawings in which like reference numerals designate identical or corresponding elements in each of the several views. However, it is to be understood that aspects of the disclosure included herein are merely exemplary of the disclosure and may be embodied in various forms. Well-known functions or constructions are not described in detail to avoid obscuring the disclosure in unnecessary detail. Therefore, specific structural and functional details disclosed herein are not to be interpreted as limiting, but merely as a basis for the claims and as a representative basis for teaching one skilled in the art to variously employ the disclosure in virtually any appropriately detailed structure.

In this description, the term “proximal” is used generally to refer to that portion of the device that is closer to a clinician during use of the device for its intended purpose, while the term “distal” is used generally to refer to that portion of the device that is farther from the clinician during use of the device for its intended purpose. In addition, the term “clinician” is used generally to refer to medical personnel including doctors, nurses, and support personnel, and directional terms such as front, rear, upper, lower, top, bottom, and similar terms are used to assist in understanding the description and are not intended to limit the disclosure.

This disclosure is directed to a surgical stapling device that includes an adapter assembly defining a longitudinal axis, a tool assembly that is pivotably coupled to the adapter assembly about an articulation axis that is transverse to the longitudinal axis, and a drive mechanism that includes a drive beam that is pivotable with the tool assembly between the non-articulated and articulated positions. By positioning the drive beam at a location to articulate with the tool assembly, the stapling device can be fired without bending the drive beam about the articulation axis to minimize the force required to fire the stapling device.

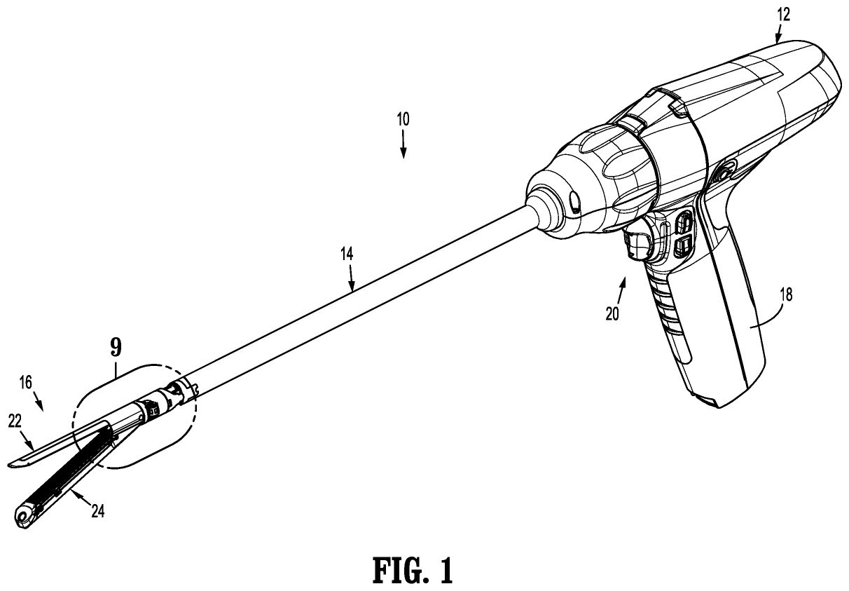

FIG. 1 illustrates a surgical stapling device shown generally as stapling device 10 that includes a handle assembly 12, an elongated body or adapter assembly 14, and a tool assembly 16. In aspects of the disclosure, the handle assembly 12 is powered and includes a stationary handgrip 18 and an actuation button or buttons 20. The actuation buttons 20 are operable to actuate various functions of the tool assembly 16 via the adapter assembly 14 including approximation, stapling, cutting of tissue, and articulation of the tool assembly 16. Although not shown, the handle assembly 16 can support batteries (not shown) that provide power to the handle assembly 12 to actuate the stapling device 10.

The tool assembly 16 is supported on a distal portion of the adapter assembly 14 and includes an anvil 22 and a cartridge assembly 24. Although the stapling device 10 is illustrated as an electrically powered stapling device, it is envisioned that the advantages of this disclosure are suitable for use with manually powered surgical stapling devices. In addition, it is contemplated, and within the scope of the disclosure for the stapling device to be configured for use with various electromechanical and/or electrosurgical instruments and systems. For example, the stapling device may be utilized in robotic surgical systems, such as the robotic surgical system shown and described in U.S. Pat. No. 8,828,023, the entire content of which is incorporated herein by reference.

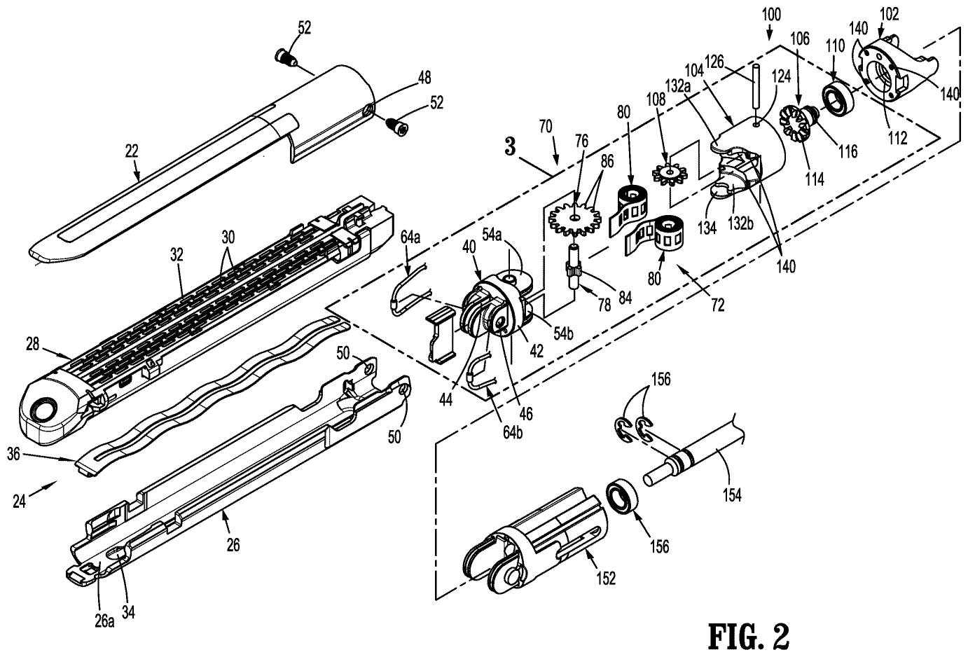

FIG. 2 illustrates the cartridge assembly 24 of the stapling device 10 (FIG. 1) which includes a channel member 26 and a staple cartridge 28 that is supported within the channel member 26. The staple cartridge 28 defines staple receiving pockets 30 (FIG. 2) and a knife slot 32. The staple receiving pockets 30 are aligned in rows on opposite sides of the knife slot 32 and receive staples (not shown). The channel member 26 also defines a knife slot 34 that is longitudinally aligned with the knife slot 32 in the staple cartridge 28. In aspects of the disclosure, the cartridge assembly 24 includes a biasing member 36 that is supported on a bottom wall 26a of the channel member 26 and supports the staple cartridge 28. The biasing member 36 is compressible or deformable to facilitate vertical movement of the staple cartridge 28 within channel member 26 to accommodate tissues of varying thicknesses. The biasing member 36 can be in the form of a wave spring or a compliant pad, although the use of other compressible or deformable materials is envisioned.

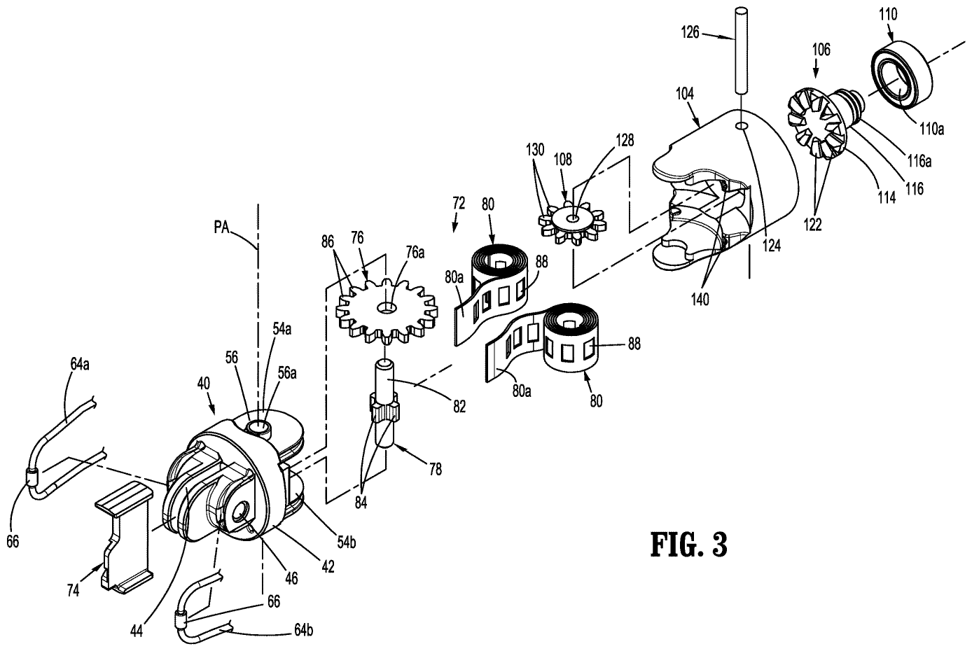

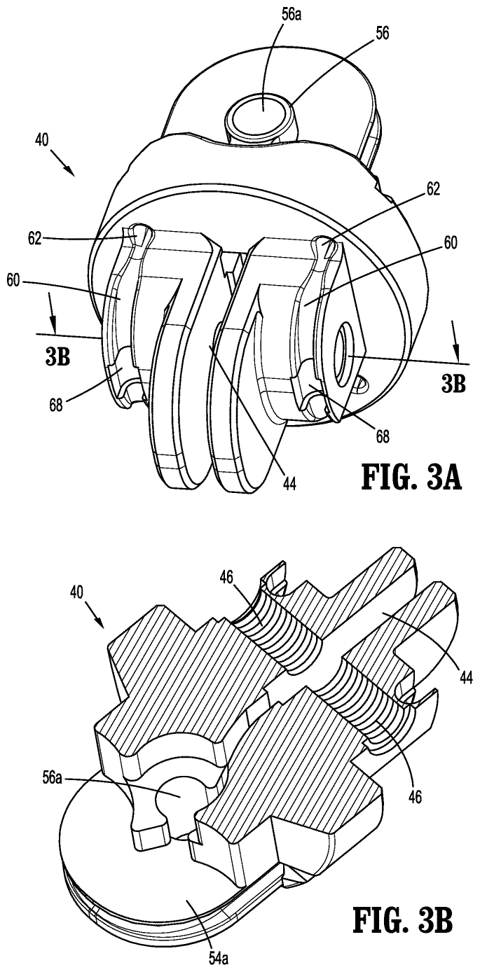

The tool assembly 16 is fixedly secured to a pivot member 40 (FIG. 3) that is positioned proximally of and secured to the tool assembly 16. The pivot member 40 includes a body 42 that defines a longitudinal slot 44 and threaded bores 46. The longitudinal slot 44 extends through the body 42 of the pivot member 40 and the threaded bores 46 are positioned on opposite sides of the longitudinal slot 44. In aspects of the disclosure, the proximal portion of the anvil 22 defines openings 48 and the proximal portion of the channel member 26 of the cartridge assembly 22 defines openings 50. The openings 48 and 50 receive screws 52 that fixedly secure the anvil 22 to the pivot member 40 and pivotably secure the channel member 26 to the pivot member 40 to facilitate movement of the channel member 26 in relation to the anvil 22 between an unclamped position and a clamped position.

FIGS. 3-3B illustrate the pivot member 40 which is secured to the proximal portion of the tool assembly 16 (FIG. 1) and includes the body 42. The body 42 of the pivot member 40 (FIG. 3B) includes proximally extending hinges having an upper hinge 54a and a lower hinge 54b as viewed in FIG. 3. Each of the upper and lower hinges 54a, 54b includes a circular pivot 56 that extends outwardly from the respective hinge 54a, 54b and defines a bore 56a.

The body 42 of the pivot member 40 also defines curved tracks 60 (FIG. 3A) that are positioned on opposite sides of the longitudinal slot 44. The curved tracks 60 are aligned with upper and lower guide bores 62 (FIG. 3A) that extend through the proximal portion of the body 42 of the pivot member 40 and receive articulation cables 64a, 64b that are positioned on opposite sides of the longitudinal slot 44. The articulation cables 64a, 64b extend about the respective curved tracks 60, through the upper and lower guide bores 62, and proximally through the adapter assembly 14 to the handle assembly 12 (FIG. 1) to facilitate articulation of the pivot member 40 and tool assembly 16 in relation to the adapter assembly 14. In aspects of the disclosure, the articulation cables 64a, 64b support ferrules 66 (FIG. 3) that are received within slots 68 (FIG. 3) formed along the curved tracks 60 to secure the articulation cables 64a, 64b to the pivot member 40.

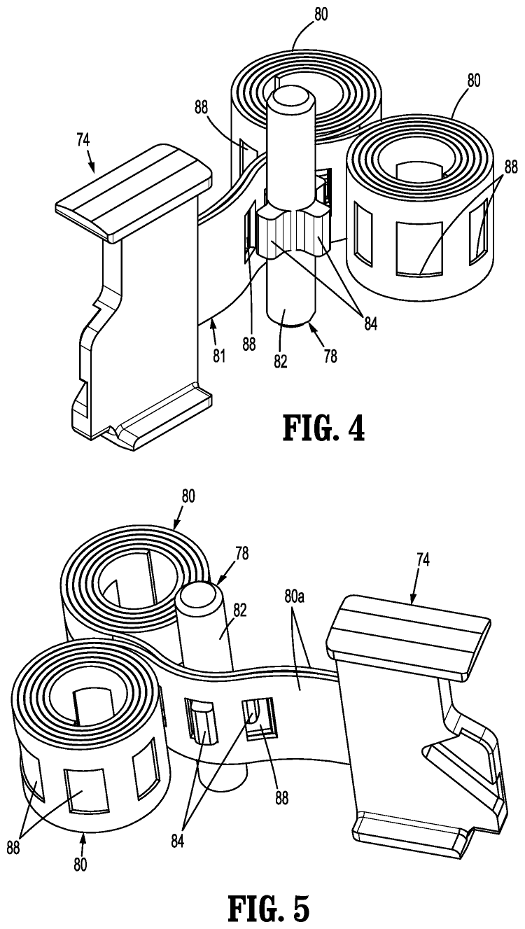

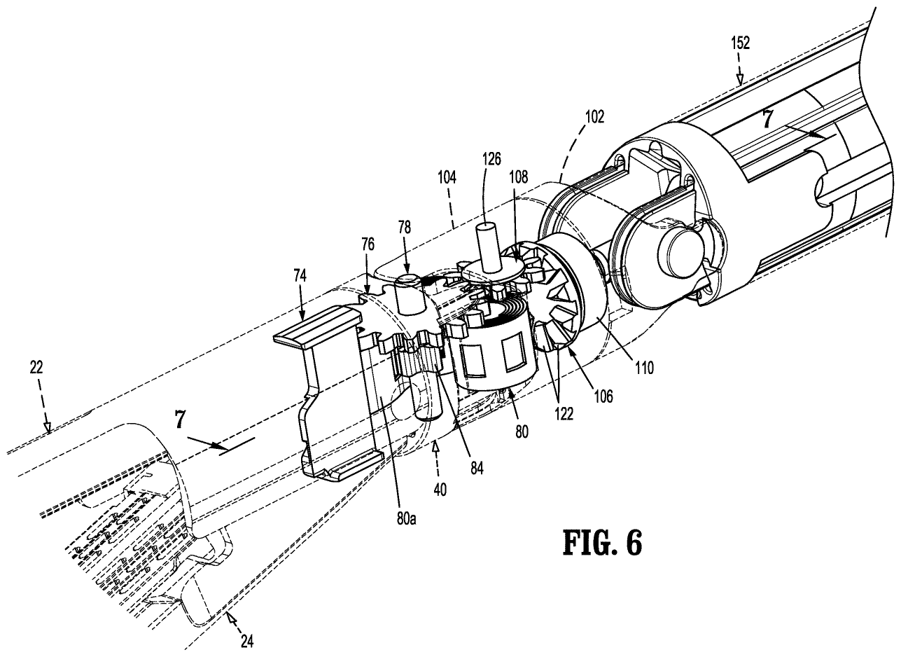

FIGS. 3-7 illustrate a distal drive assembly 72 of a drive mechanism 70 (FIG. 2) of the stapling device 10 (FIG. 1). The distal drive assembly 72 is supported on the pivot member 40 and includes a clamp member 74, a sprocket gear 76, a sprocket 78, and one or more coiled bands 80. The sprocket gear 76 defines a central bore 76a and includes a plurality of gear teeth 86 positioned about the circumference of the sprocket gear 76. The sprocket 78 includes a shaft 82 and fingers 84 positioned about a central portion of the shaft 82. The shaft 82 of the sprocket 78 is fixedly received within the central bore 76a of the sprocket gear 76 and rotatably received within the bores 56a (FIG. 3A) defined by the pivots 56 on the upper and lower hinges 54a, 54b of the pivot member 40. When the sprocket gear 76 is rotated, the sprocket 78 rotates to rotate the fingers 84 about an axis defined by the shaft 82 of the sprocket 78. A distal end of each of the coiled bands 80 is secured to the clamp member 74 such that the clamp member 74 is advanced through the tool assembly 16 (FIG. 2) when the coiled bands 80 are uncoiled and advanced by the sprocket 78.

The coiled bands 80 are supported on opposite sides of the sprocket 78 between the upper and lower hinges 54a, 54b of the pivot member 40. Each of the coiled bands 80 is elongated and defines openings 88 that are spaced along a longitudinal axis of the coiled band 80. The coiled bands 80 include distal portions 80a that are positioned side-by-side to each other to form a drive beam 81 with the openings 88 in the coiled bands 80 aligned with each other. By positioning the coiled bands 80 in side-by-side relation, rigidity is provided to the drive beam 81 as the clamp member 74 is advanced through the tool assembly 16. The openings 88 of the coiled bands 80 receive the fingers 84 of the sprocket 78 such that rotation of the sprocket 78 advances or retracts the distal portions of the coiled bands 80 through the longitudinal slot 44 defined by the pivot member 40. The sprocket 78 moves the coiled bands 80 from a coiled configuration to an extended configuration to advance the clamp member through the tool assembly 16. Although two coiled bands 80 are shown, it is envisioned that one or more coiled bands 80 could be provided to form the drive beam 81.

The clamp member 74 has an I-shaped configuration and includes a first beam 90, a second beam 92, and a vertical strut 94 that fixedly connects the first beam 90 to the second beam 92. The first beam 90 is positioned to engage the anvil 22 (FIG. 2) and the second beam 92 is positioned to engage the channel member 26 of the cartridge assembly 24 as the drive mechanism 70 is moved from a drive retracted position towards a drive advanced position to move the tool assembly 16 (FIG. 1) from the unclamped position to the clamped position.

When the drive mechanism 70 is in a retracted position, the clamp member 74 is positioned adjacent to the pivot member 40 within the proximal portion of the tool assembly 16 (FIG. 1) and the tool assembly 16 is in the unclamped position. When the sprocket 78 is rotated to rotate the fingers 84 of the sprocket 78 sequentially into the openings 88 of the coiled bands 80, the distal portions of the coiled bands 80 which define the drive beam 81 advance the clamp member 74 from a retracted position towards an advanced position to initially move the tool assembly 16 from the unclamped position to the clamped position, and to subsequently advance an actuation sled 96 (FIG. 12) within the cartridge assembly 24 to eject staples (not shown) from the staple cartridge 28 (FIG. 2). Although not described in detail, the clamp member 74 or the actuation sled 96 can support or include a knife or cutting edge to cut tissue clamped between the anvil 22 and the staple cartridge 28 as the clamp member 74 moves from the retracted position towards the advanced position.

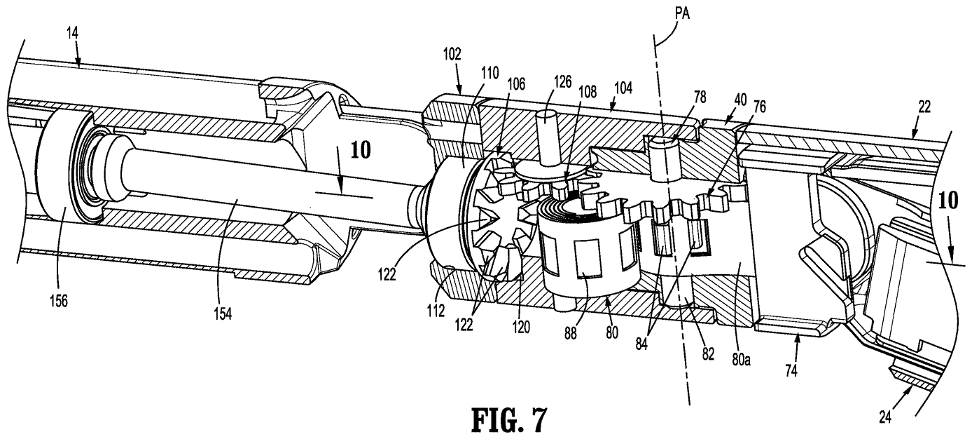

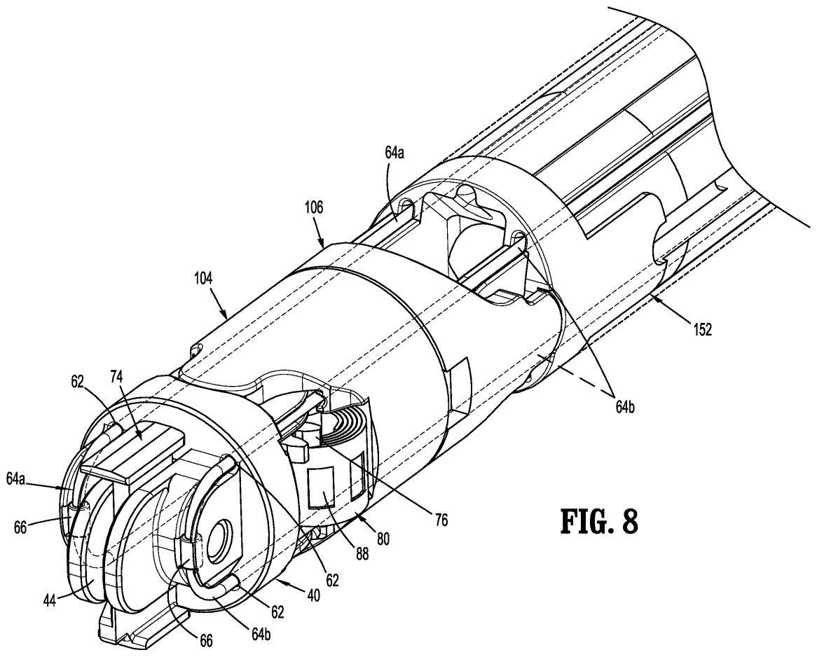

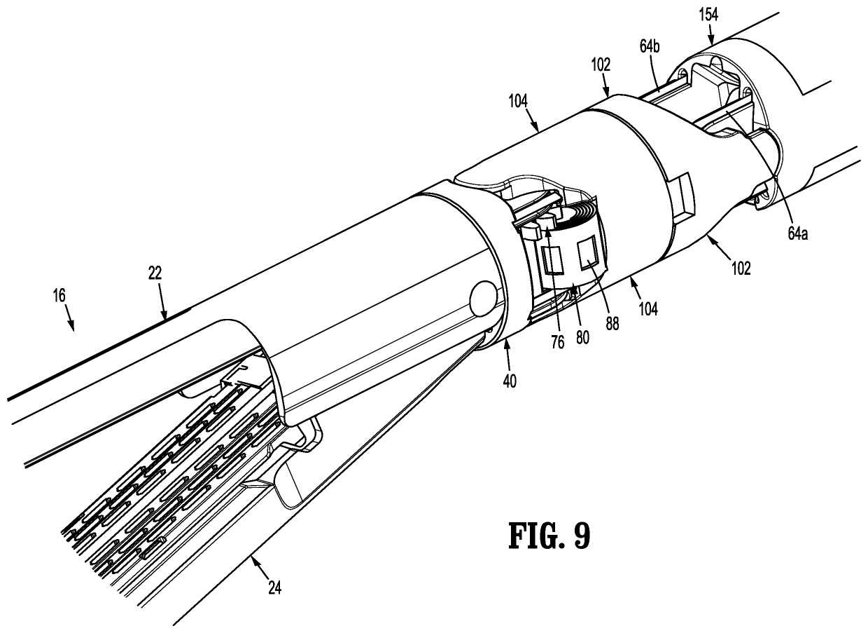

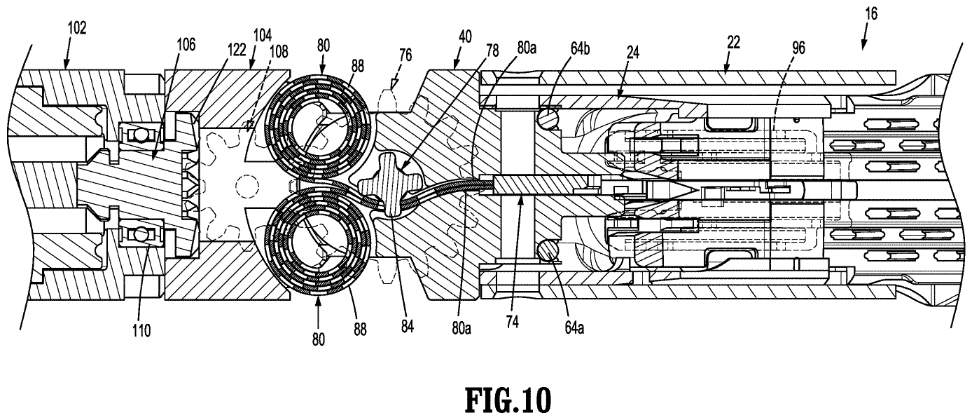

FIGS. 2, 3, and 6-8 illustrate a proximal drive assembly 100 of the drive mechanism 70 of the stapling device 10. The proximal drive assembly 100 forms the distal portion of the adapter assembly 14 (FIG. 1) and includes a proximal housing portion 102, a distal housing portion 104, a drive gear 106, a proximal gear 108, and a bearing 110. The proximal housing portion 102 of the proximal drive assembly 100 defines a stepped through bore 112 (FIG. 2) that receives the bearing 110. The drive gear 106 includes a gear plate 114 and a shaft portion 116 that extends proximally from the gear plate 114 through a bore 110a defined by the bearing 110. The shaft portion 116 of the drive gear 106 is rotatably supported on the proximal housing portion 102 by the bearing 110 and extends proximally from the stepped through bore 112 of the proximal housing portion 102 of the proximal drive assembly 100. The gear plate 114 of the drive gear 106 is received within a cavity 120 (FIG. 7) defined by the distal housing portion 104 of the proximal drive assembly 100 of the drive mechanism 70 and includes distally facing teeth 122. The distal housing portion 104 of the proximal drive assembly 100 also defines a bore 124 (FIG. 3) that extends through the distal housing portion 104 and communicates with the cavity 120.

The proximal gear 108 of the proximal drive assembly 100 is rotatably supported within the cavity 120 of the distal housing portion 104 of the proximal drive assembly 100 about an axle 126 that extends through the bore 124 defined in the distal housing portion 104. The proximal gear 108 defines a bore 128 that receives the axle 126 and includes gear teeth 130 that are positioned about an outer circumference of the proximal gear 108. The gear teeth 130 are engaged with the teeth 122 of the drive gear 106 such that rotation of the drive gear 106 causes rotation of the proximal gear 108.

The distal housing portion 104 of the proximal drive assembly 100 includes upper and lower flanges 132a, 132b that are spaced from each other and are positioned on outer surfaces of the upper and lower flanges 54a, 54b when the proximal drive assembly 100 is coupled to the pivot member 40. Each of the upper and lower flanges 132a, 132b of the distal housing portion 104 includes an inner surface that defines a circular cutout 134 that receives one of the circular pivots 56 of the upper and lower flanges 54a, 54b of the pivot member 40. Receipt of the circular pivots 56 within the circular cutouts 134 of the upper and lower flange flanges 132a, 132b of the distal housing portion 104 of the proximal drive assembly 100 pivotably couples the pivot member 40 to the proximal drive assembly 100 of the drive mechanism 70 with the shaft 82 of the sprocket 78 and the circular pivots 56 defining the articulation axis “PA” (FIG. 7).

The proximal and distal housing portions define upper and lower guide bores 140 (FIG. 2) that facilitate passage of the articulation cables 64a, 64b (FIG. 2) through the proximal drive assembly 100 into the adapter assembly 14 (FIG. 1). Although not shown, a proximal portion of the articulation cables 64a, 64b is secured to an articulation control mechanism supported within the adapter 14 or handle assembly 12 to control articulation of the tool assembly 16. Tension in the articulation cables 64a, 64b retains the circular pivots 56 of the pivot member 40 within the circular cutouts 134 of the upper and lower flanges 132a, 132b of the distal housing portion 104 of the proximal drive assembly 100 to pivotably couple the proximal drive assembly 100 to the pivot member 40. As described above, the tool assembly 16 (FIG. 1) is secured to the pivot member 40 such that the tool assembly 16 is pivotably supported on the distal portion of the adapter assembly 14 (FIG. 1) about the articulation axis “PA”. When the pivot member 40 is coupled to the proximal drive assembly 100, the teeth 130 of the proximal gear 108 of the proximal drive assembly 100 engage the gear teeth 86 of the sprocket gear 76 of the distal drive assembly 72 such that rotation of the proximal gear 108 causes rotation of the sprocket gear 76.

FIGS. 1, 2, and 7 illustrate components of the adapter assembly 14 (FIG. 1) of the stapling device 10 which include an outer tube 150 (FIG. 1), a coupling member 152, a drive member 154, a bearing 156, and C-clips 158. The coupling member 152 is secured to the proximal housing 102 portion of the proximal drive assembly 100 to secure the adapter assembly 14 (FIG. 1) to the tool assembly 16. The drive member 154 is rotatably supported by the bearing 156 within the coupling member 152 and includes a distal portion that is coupled to the shaft 116 of the drive gear 106 such that rotation of the drive member 154 causes rotation of the drive gear 106. In aspects of the disclosure, the bearing 156 is secured to the proximal portion of the drive member 154 by the C-clips 208. When the drive gear 106 is rotated by the drive member 154, the drive gear 106 rotates the proximal gear 108. As described above, the teeth 130 of the proximal gear 108 are engaged with the gear teeth 86 of the sprocket gear 76 such that rotation of the proximal gear 108 causes rotation of the sprocket gear 76 to rotate the sprocket 78. Rotation of the sprocket 78 advances and retracts the coiled bands 80 to advance and retract the clamp member 74 within the tool assembly 16. In aspects of the disclosure, the drive member 154 can include a flexible shaft.

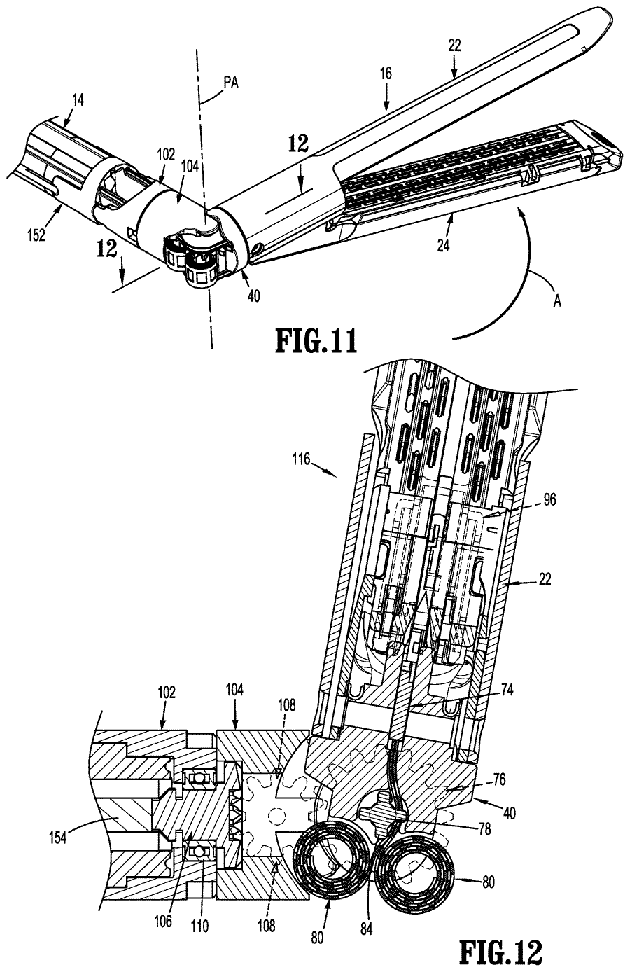

FIGS. 11 and 12 illustrate the stapling device 10 (FIG. 1) as the tool assembly 16 is moved to the articulated position. When the articulation cables 64a and 64b are retracted and advanced, respectively, the pivot member 40 which supports the tool assembly 16 pivots in the direction indicated by arrow “A” in FIG. 11 about the articulation axis “PA” from the non-articulated position to an articulated position. When the tool assembly 16 is in an articulated position, the distal drive assembly 72 of the drive mechanism 70 pivots with the tool assembly 16 about the articulation axis “PA”. As illustrated, when the tool assembly 16 is in an articulated position, the proximal gear 108 of the proximal drive assembly 100 remains engaged with the sprocket gear 76 of the distal drive assembly 72 such that rotation of the proximal gear 108 causes rotation of the sprocket gear 76.

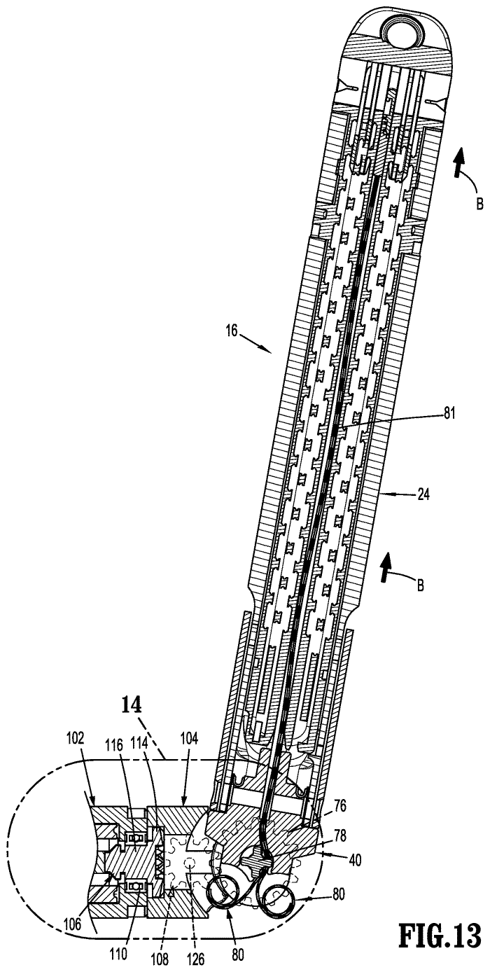

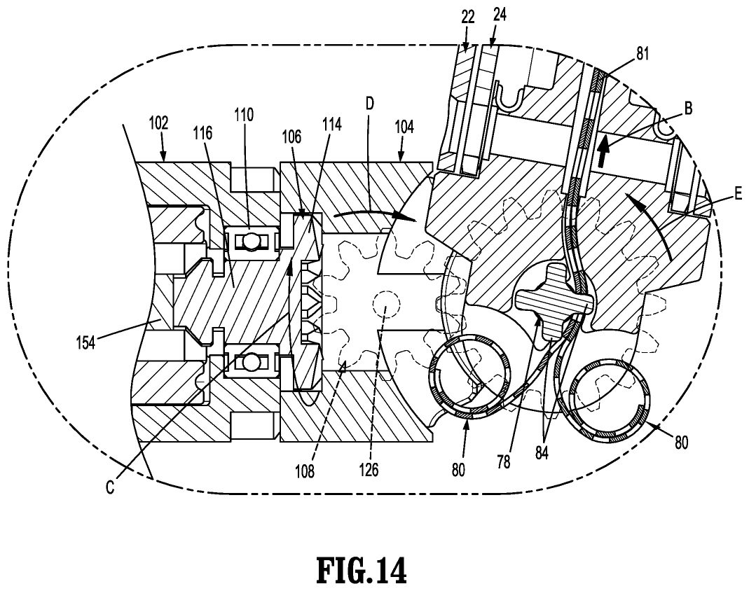

FIGS. 13 and 14 illustrate the tool assembly 16 of the stapling device 10 (FIG. 1) as the stapling device 10 is fired and the distal portion of the coiled bands 80 are advanced to advance the clamp member 74 through the tool assembly 16 in the direction indicated by arrows “B”. When the clamp member 74 is advanced within the tool assembly 16, the clamp member 74 engages the actuation sled 96 of the staple cartridge 28 to advance the actuation sled 96 within a body of the staple cartridge 28 and eject staples (not shown) from the staple cartridge 28. As described above, the fingers 84 of the sprocket 78 are received within the openings 88 of the coiled bands 80. When the drive gear 106 is rotated in the direction of arrow “C” in FIG. 14, the proximal gear 108 rotates in the direction of arrow “D”, and the sprocket gear 76 rotates in the direction indicated by arrow “E” to rotate the sprocket 78. As the sprocket 78 rotates, the fingers 84 of the sprocket 78 are received sequentially within the openings 88 of the coiled bands 80 to advance the coiled bands 80 through the longitudinal slot 44 of the pivot member 40. As the coiled bands 80 are advanced, the two bands 80 form the drive beam 81 to advance the clamp member 74 through the tool assembly 16.

After the stapling device 10 (FIG. 1) is fired and the clamp member 74 is in the advanced position, the drive member 154 can be rotated in an opposite direction to rotate the sprocket 78 in the opposite direction to retract and recoil the bands 80. The drive mechanism 70 described herein positions the drive beam 81 distally of the articulation axis “PA” defined between the tool assembly 16 and the adapter assembly 14. Thus, the drive beam 81 formed by the coiled bands 80 do not have to bend about the articulation axis “PA” during firing to minimize the force required to fire the stapling device 10 (FIG. 1).

Persons skilled in the art will understand that the devices and methods specifically described herein and illustrated in the accompanying drawings are non-limiting exemplary embodiments. It is envisioned that the elements and features illustrated or described in connection with one exemplary embodiment may be combined with the elements and features of another without departing from the scope of the disclosure. Also, one skilled in the art will appreciate further features and advantages of the disclosure based on the above-described embodiments. Accordingly, the disclosure is not to be limited by what has been particularly shown and described, except as indicated by the appended claims.

What is claimed is:

1. A surgical stapling device comprising:
an adapter assembly having a distal portion supporting a proximal drive assembly including a drive gear and a proximal gear that is engaged with the drive gear;
a tool assembly including an anvil and a cartridge assembly, the anvil and the cartridge assembly coupled to each other to facilitate movement of the tool assembly between an unclamped position and a clamped position, the tool assembly including a proximal portion and a distal portion; and
a distal drive assembly secured to the proximal portion of the tool assembly, the distal drive assembly including a pivot member, a sprocket, a sprocket gear, a coiled band having a distal portion, and a clamp member secured to the distal portion of the coiled band, the coiled band movable in response to rotation of the sprocket between a coiled configuration and an extended configuration to move the clamp member in relation to the tool assembly between retracted and advanced positions.

2. The surgical stapling device of claim 1, wherein the sprocket includes fingers, and the coiled band defines openings that receive the fingers of the sprocket such that rotation of the sprocket in a first direction causes advancement of the distal portion of the coiled band to advance the clamp member in relation to the tool assembly.

3. The surgical stapling device of claim 1, wherein the coiled band includes first and second coiled bands.

4. The surgical stapling device of claim 3, wherein the pivot member defines a longitudinal slot that is longitudinally aligned with the distal portion of the first and second coiled bands.

5. The surgical stapling device of claim 1, wherein the pivot member is pivotably coupled in relation to the proximal drive assembly about an articulation axis such that the coiled band pivots about the articulation axis with the pivot member.

6. The surgical stapling device of claim 2, further including first and second articulation cables extending through the adapter assembly and coupled to the pivot member, the first and second articulation cables movable to pivot the tool assembly about the articulation axis.

7. The surgical stapling device of claim 6, wherein the proximal drive assembly includes a distal housing portion that defines circular cutouts, and the pivot member includes circular pivots that are received within the circular cutouts to pivotably couple the pivot member to the proximal drive assembly.

8. The surgical stapling device of claim 7, wherein the first and second articulation cables extend through channels in the proximal drive assembly and the pivot member, and wherein tension in the first and second articulation cables retains the circular pivots within the circular cutouts.

9. The surgical stapling device of claim 1, wherein the proximal drive assembly includes a proximal gear that is engaged with the drive gear, the proximal gear engaged with the sprocket gear such that rotation of the proximal gear causes rotation of the sprocket gear.

10. The surgical stapling device of claim 3, wherein the first and second bands are positioned side-by-side as the distal portions of the first and second bands pass through the longitudinal slot of the pivot member to form a substantially rigid drive beam.

11. The surgical stapling device of claim 1, further including a handle assembly, the proximal portion of the adapter assembly coupled to the handle assembly.

12. The surgical stapling device of claim 5, wherein the distal drive assembly pivots with the tool assembly between the non-articulated position and the articulated positions.

13. A surgical stapling device comprising:
a housing;
an adapter assembly having a proximal portion and a distal portion, the proximal portion of the adapter assembly coupled to the housing;
a tool assembly including a first jaw and a second jaw, the first jaw and the second jaw coupled to each other to facilitate movement of the tool assembly between an unclamped position and a clamped position, the tool assembly including a proximal portion; and
a distal drive assembly secured to the proximal portion of the tool assembly and including a clamp member, a coiled band, and a pivot member pivotably coupled to the distal portion of the adapter assembly, the pivot member defining a longitudinal slot, the coiled band supported on the pivot member and including a distal portion that extends through the longitudinal slot, the coiled band movable between a coiled configuration and an extended configuration to move the clamp member in relation to the tool assembly between retracted and advanced positions.

14. The surgical stapling device of claim 13, wherein the distal drive assembly includes a sprocket having fingers and the coiled band defines openings that receive the fingers such that rotation of the sprocket in a first direction causes advancement of coiled band from the coiled configuration to the extended configuration to advance the clamp member in relation to the tool assembly.

15. The surgical stapling device of claim 14, further including a proximal drive assembly supported on the distal portion of the adapter assembly, the proximal drive assembly including a distal housing portion that defines circular cutouts, the pivot member including circular pivots that are received within the circular cutouts to pivotably couple the pivot member to the proximal drive assembly.

16. The surgical stapling device of claim 15, wherein first and second articulation cables extend through channels in the proximal drive assembly and the pivot member, and wherein tension in the first and second articulation cables retains the circular pivots within the circular cutouts.

17. The surgical stapling device of claim 13, wherein the coiled band includes first and second coiled bands, each of the first and second coiled bands having a distal portion that is secured to the clamp member.

18. The surgical stapling device of claim 17, wherein the distal portions of the first and second bands are positioned side-by-side as the distal portions of the first and second bands pass through the longitudinal slot of the pivot member to form a substantially rigid drive beam.

19. The surgical stapling device of claim 18, wherein the distal drive assembly pivots with the tool assembly between the non-articulated position and the articulated positions.

20. A surgical stapling device comprising:
an elongated shaft including a distal end;
a tool assembly positioned at the distal end of the elongated shaft, the tool assembly including a first jaw and a second jaw, the first jaw and the second jaw coupled to each other and moveable between an unclamped position and a clamped position, and
a drive assembly including a clamp member, at least one coiled band coupled to the clamp member, and a pivot member, the at least one coiled band movable between a coiled configuration and an extended configuration to move the clamp member in relation to the tool assembly between retracted and advanced positions.
